(19) United States
(12) Patent Application Publication
(10) Pub. No.: US 2009/0152137 A1
(43) Pub. Date: Jun. 18, 2009

(54) Combined Skin Sealant And Incise Drape

(76) Inventors: Thomas Gregory Estes, Atlanta, GA (US); Katherine Luann Dziak, Cumming, GA (US); Cherie Everhart, Alpharetta, GA (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/956,569

(22) Filed: Dec. 14, 2007

(51) Int. Cl. A61B 19/08; B65D 69/00

(52) U.S. Cl. 206/232; 128/849; 206/570

(57) ABSTRACT

A combination drape which has both a skin sealant and an incise drape is applied to a patient's skin. The skin sealant is positioned adjacent the prepped skin and the incise drape is positioned over the skin sealant and the prepped skin. The skin sealant substantially fills contours of the incise drape positioned thereover such that any air pockets and wrinkles positioned between the incise drape and the skin sealant are sealed and isolated from the skin. The combination drape is contained within a kit which desirably includes instructions.

### BACKGROUND

Surgical site infections (SSI) occur following about 2-3 percent of surgeries in the United States with an estimated 500,000 incidents of SSI occurring annually, which can lead to significant patient morbidity and mortality. In addition to the negative impact of such infections on patient health, these potentially avoidable infections contribute significantly to the financial burden experienced by the health care system. SSIs result when an incision becomes contaminated by bacteria, and for most surgeries the primary source of these infection-causing microorganisms is the skin (an exception being surgeries in which the gastrointestinal tract is penetrated).

Various compositions are used to prepare the skin prior to surgery. Skin preparations or “preps” are used to remove some level of microbial load on the skin prior to making an incision. Skin preps are applied to the skin and allowed to dry to maximize effectiveness for reducing microorganisms. After the skin prep has dried, a clear drape may be positioned over the area of the skin in which an incision will be made. Referred to as an “incise drape,” such a drape frequently also has an antimicrobial agent formed therewith and/or applied thereto, to reduce the introduction of microorganisms into the incision.

Such skin preps currently are predominantly povidone-iodine or chlorhexidine gluconate based formulations and may contain alcohol for fast drying and more effective killing of organisms. Time constraints in the operating room may result in the skin remaining wet when draping and/or surgery begin, creating the possibility of infection. Drawback are present in the incise drape as well.

The incise drape is clear, flexible, and has an adhesive surface. When it is applied to a patient, the incise drape conforms to and adhesively seals against a patient's skin. However, it is common to see a plurality of small pockets of air that are trapped between the incise drape and the patient's skin. Such air pockets permit undesirable microorganisms to contaminate in and around the incision.

Skin sealant materials are used to protect patients from bacterial infections associated with surgical site incisions and insertion of intravenous needles. Skin preps are applied to the skin and allowed to dry to maximize effectiveness for reducing microorganisms. After the skin prep has dried, the sealant may be applied directly to the skin in liquid form. The sealant forms a coherent film with strong adhesion to the skin through various techniques based on the chemistry of the sealant composition.

Skin sealants now use a polymer composition that dries to form a film through evaporation of a solvent, for example. Other skin sealants contain monomeric units that polymerize in situ to from a polymeric film. Cyanoacrylate sealants containing alkyl cyanoacrylate monomer are an example of the latter type wherein the monomer polymerizes in the presence of a polar species such as hydroxide, water or protein molecules to form an acrylic film. The resulting film formed serves to immobilize bacterial flora found on the skin and prevents their migration into an incision made during a surgical procedure or skin puncture associated with insertion of an intravenous needle.

Skin sealants may contain additives such as plasticizing agents to improve film flexibility and conformance, viscosity modifiers to aid in application of the liquid composition, free radical and anionic scavengers to stabilize the product prior to use, biocidal agents to kill immobilized bacteria under the film, and the like. Therefore, skin sealants often replace incise drapes due to their advantages. Such skin sealants do have disadvantages.

Drapes surrounding the skin sealant must be taped or adhesively coupled to the edge of the skin sealant. Such taping or adhesion is awkward, time consuming, and does not always assure a good seal and/or anchor of the surrounding drapes to the area of the incision. When such a seal on a drape is breached or opened, the opportunity for contamination from such an opening to migrate to the incision is present. This is unlike an incise drape, which seals over the incision, the site adjacent the incision and also to the surrounding drapes. In this manner, the incise drape acts as an anchor to secure and hold in place all of the drapes used about the surgical site.

Therefore, there is a need to combine the advantages of a skin sealant with the advantages of an incise drape. Such combination, however, necessarily needs to preserve the advantages while substantially removing the disadvantages.

### SUMMARY OF THE INVENTION

In response to the difficulties and problems discussed herein, a combination drape is provided. The drape includes a skin sealant and an incise drape. The skin sealant is disposed on the incise drape and the combination skin sealant and incise drape is applied to a patient's skin such that the skin sealant is positioned adjacent the skin and the incise drape is positioned over the skin sealant and the skin. The skin sealant substantially fills contours of the incise drape positioned thereover such that any air pockets and wrinkles positioned between the incise drape and the skin sealant are sealed and isolated from the skin.

In another aspect of the invention, a kit for draping an incision site is provided. The kit includes a combination drape, including a skin sealant and an incise drape. The kit also includes Instructions. The skin sealant is disposed on the incise drape and the combination skin sealant and incise drape is applied to a patient's skin such that the skin sealant is positioned adjacent the skin and the incise drape is positioned over the skin sealant and the skin. The skin sealant substantially fills contours of the incise drape positioned thereover such that any air pockets and wrinkles positioned between the incise drape and the skin sealant are sealed and isolated from the skin. The instructions provide guidance on the selection and sequence of use of items in the kit.

### BRIEF DESCRIPTION OF THE DRAWINGS

FIG. 1 is a perspective view of an incise drape of the present invention, showing on a lower surface peel strips forming a reservoir;

FIG. 2 is a perspective view of the incised drape of the present invention, showing an upper surface;

FIG. 3 is a cross-sectional view of FIG. 1 taken along lines 3-3;

FIG. 4 is a cross-sectional view similar to FIG. 3, but showing thing a thicker area of the incise drape near peripheral edges, and a thinner area in a central area;

FIG. 5 is a perspective view of the combination drape of the present invention, showing the incise drape of FIG. 1 and the reservoir filled with skin sealant;

FIG. 6 is a cross-sectional view of FIG. 5 taken along lines 6-6;

FIG. 7 is a perspective view similar to FIG. 5, but showing a cover positioned over the skin sealant;

FIG. 8 is a cross-sectional view of FIG. 7 taken along lines 8-8;

FIG. 9 is a perspective view similar to FIG. 5 and FIG. 7, but showing a partially raised cover positioned over the skin sealant;

FIG. 10 is a cross-sectional view of FIG. 9 taken along lines 10-10;

FIG. 11 is a perspective view of a porous film used in conjunction with the skin sealant;

FIG. 12 is a perspective view of the present invention similar to FIG. 5, but showing in phantom lines the porous film positioned in the skin sealant;

FIG. 13 is a cross-sectional view of FIG. 12 taken along lines 13-13;

FIG. 14 is a perspective view of the present invention similar to FIG. 1, but showing a polymer film positioned in the reservoir and adhered to the adhesive therein;

FIG. 15 is a cross-sectional view of FIG. 14 taken along lines 15-15;

FIG. 16 is a cross-sectional view similar to FIG. 15, but showing the polymer film, the porous film and the skin sealant disposed in the reservoir;

FIG. 17 is a perspective view of the present invention, showing the present invention as shown in FIG. 7 provided in a package or kit along with the porous film, the polymer film, and instructions.

FIG. 18 is a perspective view of another embodiment of the present invention, showing the present invention as shown in FIG. 1 provided in a package or kit along with the skin sealant provided separately in a container, the porous film, the polymer film, and the instructions;

FIG. 19 is a perspective view of the present invention showing the combination drape positioned on a patient with the incise drape adhered to surrounding drapes to provide an anchor and stabilizing the surrounding drapes, the combination drape also positioned over contours of a patient's body and the combination drape following the contours and forming wrinkles; and

FIG. 20 is a cross-sectional view of FIG. 19 taken along lines 20-20, showing wrinkles and air pockets formed while following the contours of a patient's body and showing the skin sealant filling and/or sealing the air pockets to isolate them away from the patient's skin.

### DEFINITIONS

As used herein the following terms have the specified meanings, unless the context demands a different meaning, or a different meaning is expressed; also, the singular generally includes the plural, and the plural generally includes the singular unless otherwise indicated.

As used herein, the terms “comprise”, “comprises”, “comprising” and other derivatives from the root term “comprise” are intended to be open-ended terms that specify the presence of any stated features, elements, integers, steps, or components, but do not preclude the presence or addition of one or more other features, elements, integers, steps, components, or groups thereof. Similarly, the terms “include”, “includes”, “has” and/or “have”, and derivatives thereof, are intended to be interpreted as the word “comprise”, and are intended to be open-ended terms that specify the presence of any stated features, elements, integers, steps, or components, but do not preclude the presence or addition of one or more other features, elements, integers, steps, components, or groups thereof.

As used herein, the term “layer” when used in the singular can have the dual meaning of a single element or a plurality of elements.

As used herein the terms “bonded” and “bonding” refer to the joining, adhering, connecting, attaching, or the like of two elements. Two elements will be considered to be bonded together when they are bonded directly to one another or indirectly to one another, such as when each is directly bonded to intermediate elements. Such bonding may occur for example, by adhesive, thermal or ultrasonic methods.

As used herein, the term “film” may refer to a breathable film or a nonbreathable film. A film layer used as a portion of the fabric described herein can be formed of any film that can be suitably bonded or attached to top and/or bottom layers of the fabric or nonwoven to yield a barrier material having the unique combination of performance characteristics and features described herein. Such a film layer is desirably formed from a polymer.

As used herein, the term “contaminant” shall mean a chemical agent or biological organism/pathogen that can potentially harm a human being or animal.

As used herein, the term “liquid” refers to any liquid, fluid, or mixture of gas and liquid; various types of aerosols and particulate matter may be entrained with such liquids.

As used herein, the term “couple” includes, but is not limited to, joining, connecting, fastening, linking, tying, adhering (via an adhesive), or associating two things integrally or interstitially together.

As used herein, the term “configure” or “configuration”, and derivatives thereof means to design, arrange, set up, or shape with a view to specific applications or uses. For example: a military vehicle that was configured for rough terrain; configured the computer by setting the system's parameters.

As used herein, the terms “substantial” or “substantially” refer to something which is done to a great extent or degree; a significant or great amount; for example, as used herein “substantially” as applied to “substantially” covered means that a thing is at least 70% covered.

As used herein, the term “alignment” refers to the spatial property possessed by an arrangement or position of things in a straight line or in parallel lines.

As used herein, the terms “orientation” or “position” used interchangeably herein refer to the spatial property of a place where or way in which something is situated; for example, “the position of the hands on the clock.”

As used herein, the term “about” adjacent to a stated number refers to an amount that is plus or minus ten (10) percent of the stated number.

These terms may be defined with additional language in the remaining portions of the specification.

### DETAILED DESCRIPTION OF THE INVENTION

Reference will now be made in detail to one or more embodiments of the invention, examples of which are illustrated in the drawings. Each example and embodiment is provided by way of explanation of the invention, and is not meant as a limitation of the invention. For example, features illustrated or described as part of one embodiment may be used with another embodiment to yield still a further embodiment. It is intended that the invention include these and other modifications and variations as coming within the scope and spirit of the invention.

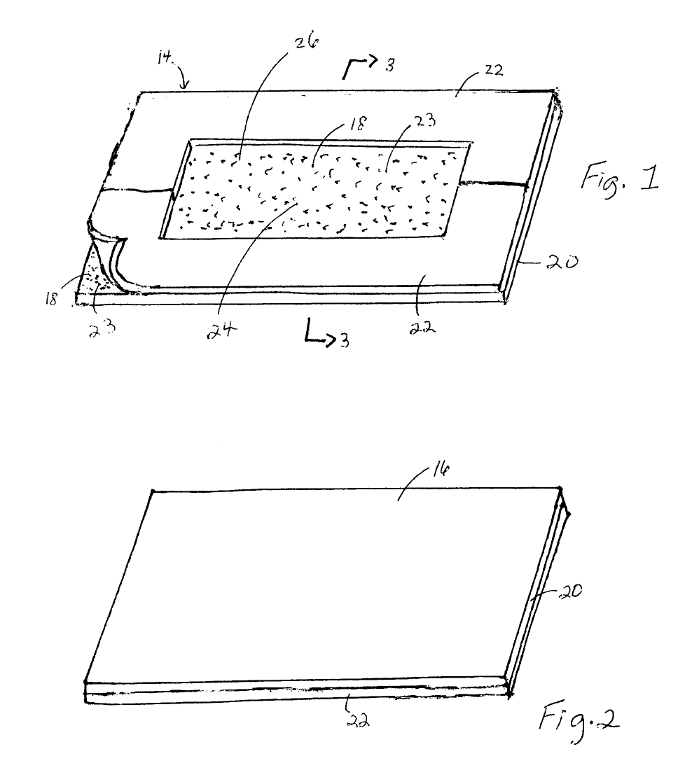

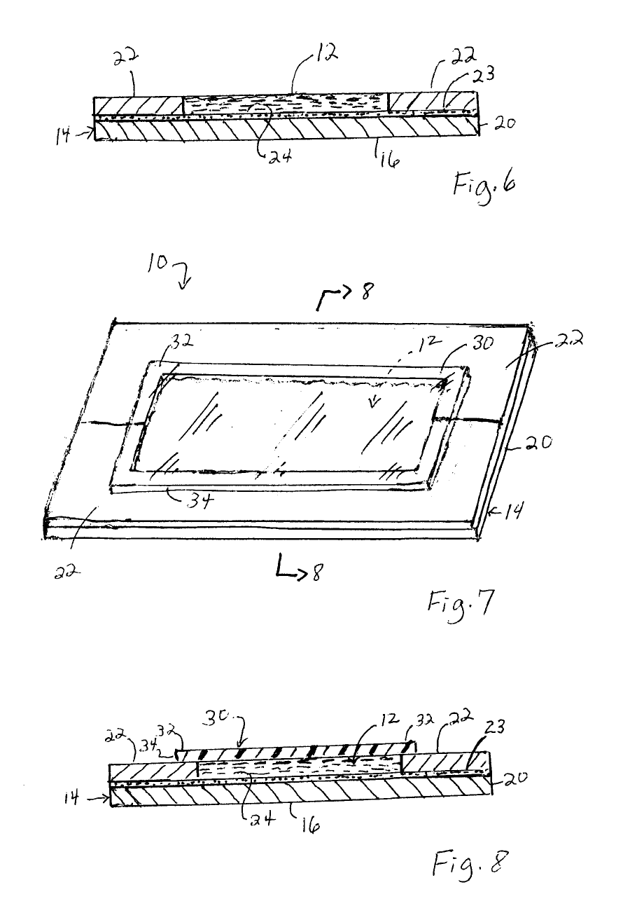

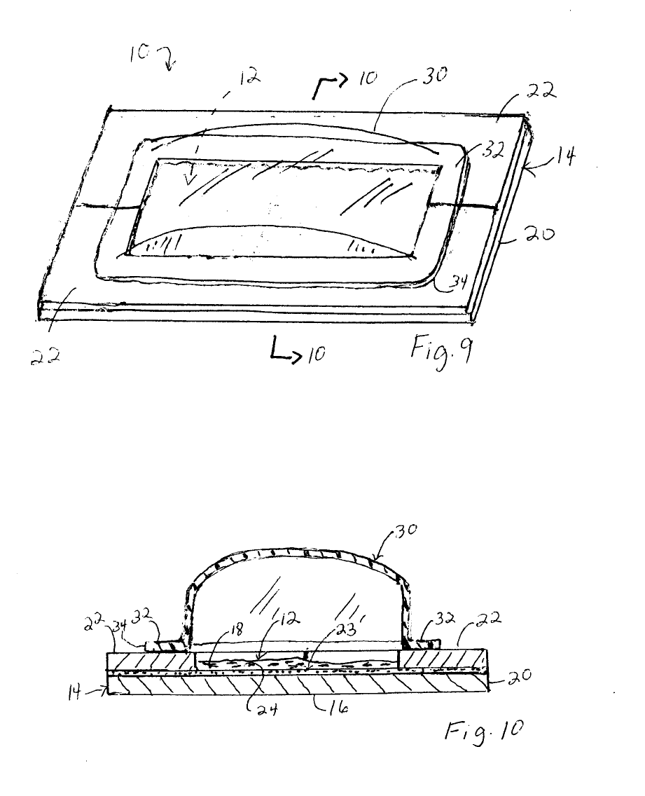

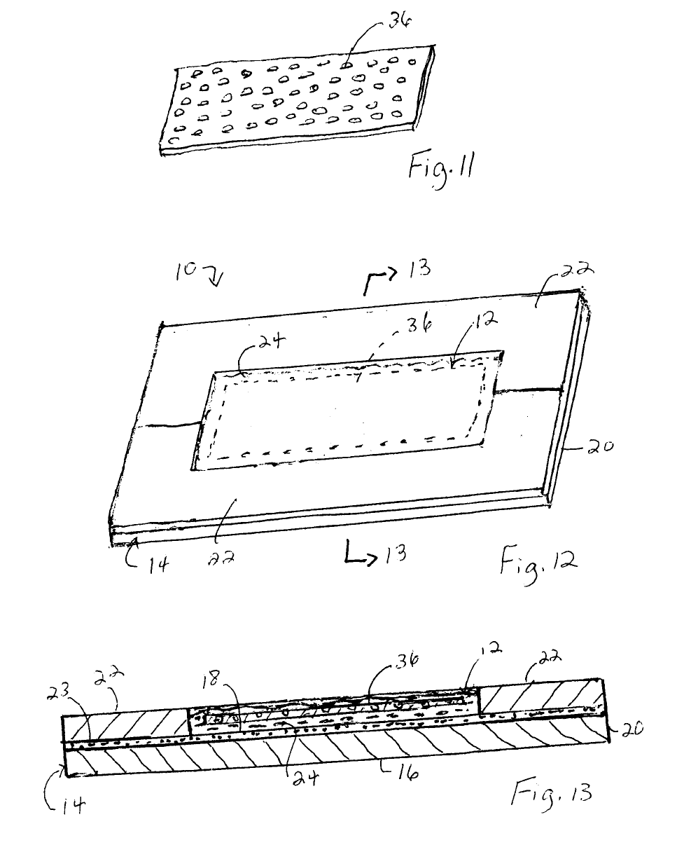

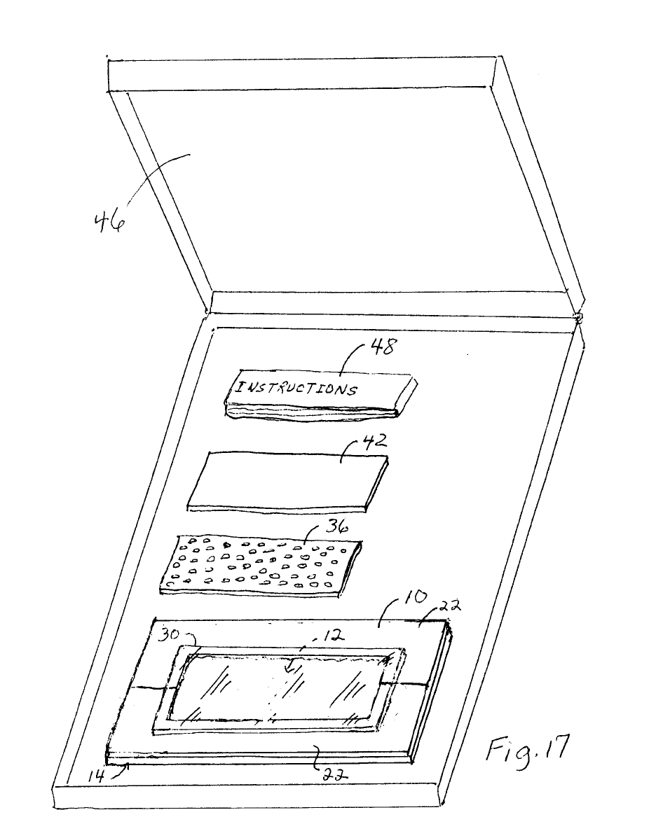

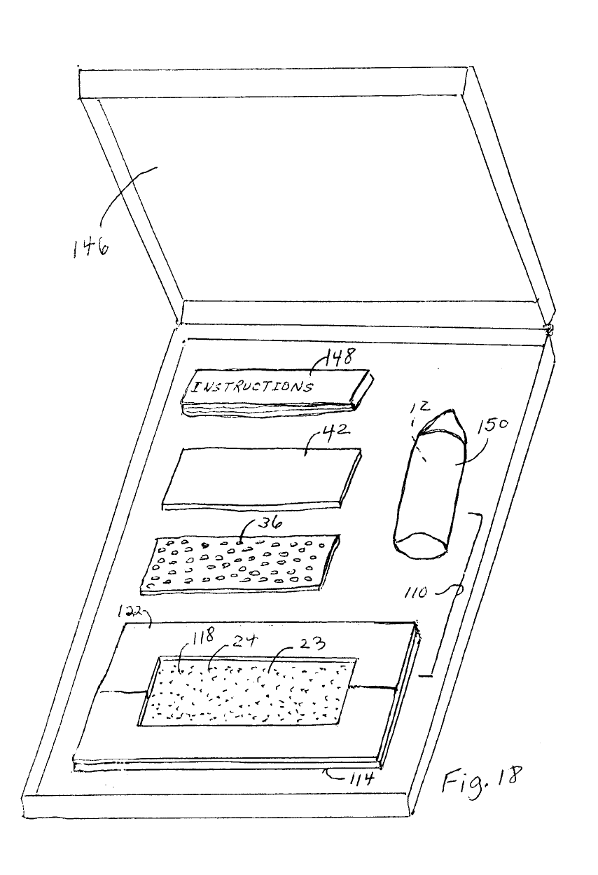

Turning now to the drawings as illustrated in FIGS. 1-20, and in particular, to FIGS. 5 and 7, combined skin sealant and incise drape 10 is provided. As noted previously herein, the skin over and in the area of where an incision will be made during a medical or surgical procedure is cleansed by the use of a skin preparation.

Skin preparations or “preps” are used to remove some level of microbial load on the skin prior to making an incision. Skin preps are applied to the skin and allowed to dry to maximize effectiveness for reducing microorganisms. Skin preps currently are predominantly povidone-iodine or chlorhexidine gluconate based formulations and may contain alcohol for fast drying and more effective killing of organisms. Povidone iodine, available commercially as Betadine® is estimated to be used in 80 percent of surgeries as a skin preparation. Betadine® skin prep is an aqueous solution of 10 percent povidone iodine having 1 percent titratable iodine content. When Betadine® skin prep is applied to the skin, it imparts an orange-brown see-through color tint. It is necessary to permit the skin prep to completely dry, before the combined skin sealant and incise drape is applied to the prepped skin. Skin sealant materials are used to protect patients from bacterial infections associated with surgical site incisions and insertion of intravenous needles.

Skin sealants are often applied directly over or on top of (Betadine®) skin preps. The sealant forms a coherent film with strong adhesion to the skin through various techniques based on the chemistry of the sealant composition. The skin sealants used herein contain a film former and a plasticizer and other optional ingredients like viscosity modifiers to aid in application of the liquid composition, free radical and anionic scavengers to stabilize the product prior to use, biocidal agents to kill immobilized bacteria under the film, and the like.

One film former available in a skin sealant composition is commercially known as InteguSeal® and is available from Medlogic Global, Ltd of Plymouth, England. InteguSeal® skin sealant contains medical grade n-butyl cyanoacrylate monomer (80% w/w). Medical grade cyanoacrylate is double distilled. Non-medical grade cyanoacrylate, in contrast, is single distilled and is typically marketed as a “super glue” type adhesive for gluing a wide variety of substrates together.

Incise drapes are often applied over or on top of (Betadine®) skin preps. Incise drapes usually have an antimicrobial agent applied to and/or formed with the drape. Incise drapes also have an adhesive applied substantially to one side of the drape, that is, the portion of the drape that is positioned against a patient's prepped skin. Such incise drapes are clear but often have a tint, such as a slightly orange-brown tint, due to the antimicrobial agent. One incise drape available is commercially known as Ioban™ and is available from 3M Corporation, St. Paul, Minn.

In the present invention, a skin sealant, such as, for example, but not by way of limitation, Integuseal® is combined with an incise drape, such as, for example, but not by way of limitation, Ioban™. The skin sealant 12 is positioned on the incise drape 14 to provide the combined skin sealant and incise drape 10 (hereinafter “combined drape” or “combination drape”).

Turning now generally to FIGS. 1-20, and more particularly to FIGS. 1-10, such a combination drape or combined drape 10 is provided. The combined drape 10 shown is generally rectangular, but it will be appreciated that the drape configuration is non-limiting, and any shape or configuration may be utilized so long as it operates as shown and/or described herein. As illustrated in FIGS. 1-6, the combined drape 10 includes the incise drape 14 which has an upper surface 16, a lower surface 18, and an outer peripheral edge 20. A pair of peel strips 22 are positioned adjacent the outer peripheral 20 of the incise drape 14, covering a portion of the adhesive 23 on the lower surface 18. The peel strips 22 cooperate to provide, prior to their removal, a shallow reservoir 24 in a central area 26 on the lower surface 18 of the drape 14. A single peel strip (not shown) may also be utilized, or the pair of peel strips 22 may be connected, and the connection broken upon removal from the drape 14 (not shown).

Alternatively, a portion 28 adjacent the outer peripheral edge 20 of the incise drape 14 which extends a distance toward the central area 26 of the incise drape 14 is formed to provide a thicker incise drape 14 near the outer peripheral edge 20, and a thinner area of the incise drape 14 is provided in the central area 26 such that the reservoir 24 is formed in the thinner area (FIG. 4). In yet another alternative, a continuous rim of material is positioned or, alternatively, formed, a distance from the peripheral edge 20 of the incise drape 14 to encircle the central area 26 and provide the reservoir 24 (not shown).

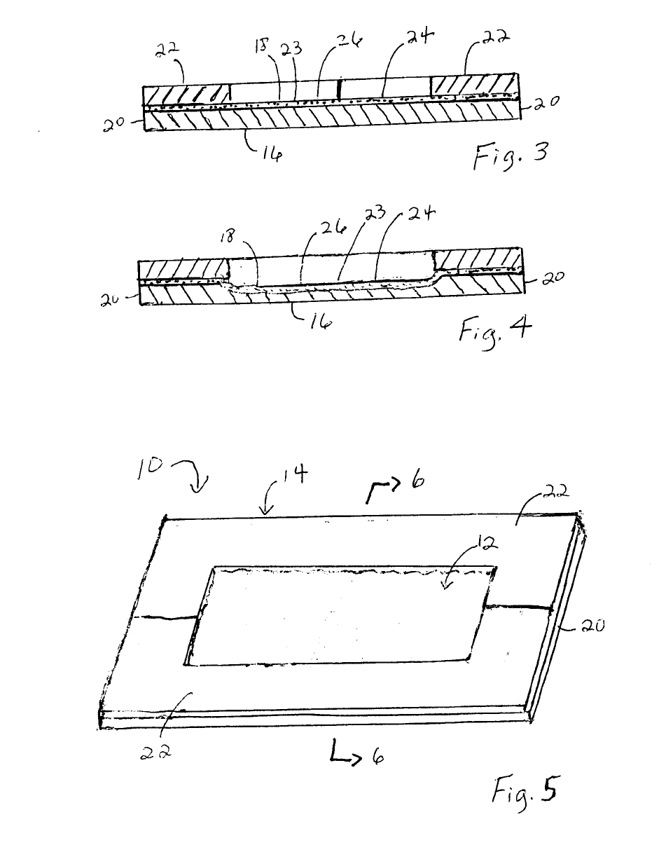

A skin sealant 12 is desirably positioned in the reservoir 24. The skin sealant 12 is held in place and maintained in a non-hardened position in the reservoir 20 of the incise drape 14 by a cover 30, as shown in FIGS. 7-10. The cover 30 may be clear and/or opaque. The cover 30 may include a lip 32 formed about an edge 34 such that the cover 30 is sealed to the incise drape 14 to maintain the skin sealant 12 in the reservoir 24. Alternatively, the cover 30 may not include a well defined lip 32. The cover 30 may be raised slightly higher relative to the peel strip(s) 22 (FIGS. 7 and 8), may be positioned horizontally at about the same elevation or just above (FIG. 3), or may have a lower elevation (not shown). A clear porous film 36, as shown in FIGS. 11-13, may be positioned within the cover 30 and within the skin sealant 12 and extend generally over at least a portion of the skin sealant 12 so that the skin sealant 12 is generally evenly distributed within the reservoir 24. Alternatively, the skin sealant 12 alone may be contained within the reservoir 24, as illustrated in FIGS. 7-10.

To apply the combined drape 10 to a patient's prepped skin 38, the cover 30 is removed to expose the skin sealant 12 (FIG. 5). The combined drape 10 is then inverted so that the lower surface 18 is positioned above the prepped skin 38, as illustrated in FIGS. 19 and 20. The combined drape 10 may be positioned sequentially against the prepped skin 38 such that the skin sealant 12 is positioned on the prepped skin 38 first, and then the peel strips 22 are removed and the remaining incise drape 14 is positioned on the patient's prepped skin 38. The peripheral edge 20 of the incise drape 14 and portions 40 of the incise drape near the edges 20 are adhered to the surrounding drapes 41 to provide an anchor to the incision site as well as to provide a complete, intact draped closure over the surgical site. There may be a slight delay in applying the incise drape 14, since this broader drape 14 may need to be positioned and molded about, around and over contours of the incision site and the adjacent area. Alternatively, however, the cover 30 is removed and the combined drape 10 is inverted so that the lower surface 18 is positioned above the prepped skin 38. The peel strips 22 are removed and the combined drape 10 (skin sealant 12 and incise drape 14) are applied substantially simultaneously to the patient's prepped skin 38.

When the surgery is nearing completion, the incise drape 14 may be removed before final sutures, staples, or other closing materials are applied to close the incision. In most instances, when the incise drape 14 is removed, the skin sealant 12 will substantially peel away from the incision area as well, thereby substantially removing the combined drape 10 (not shown). In some instance, this is a desired result.

In other instances, it may be desirable for the skin sealant 12 to remain in place about the incision site. When it is desirable to leave the skin sealant 12 in place about the area of the incision, the reservoir 24 of the incise drape 14 may be modified.

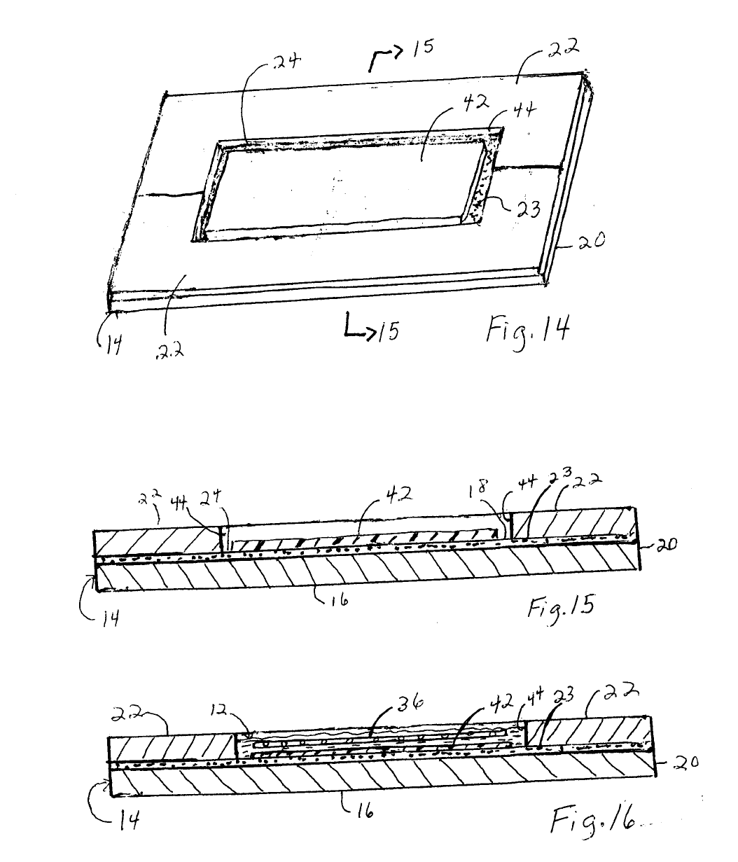

Such modification may be in the form of a thin piece of polymer film 42, shown in FIGS. 14-16, applied to the area of the reservoir 24, which is sized to cover both the site of the incision and the immediate surrounding area, but desirably does not extend beyond the area of the reservoir 24. The polymer film 42 permits the incise drape 14 to be removed, while the skin sealant 12 remains in place on the skin 38. Such a polymer film 42, for example but not by way of limitation, may include polyethylene, polypropylene, and/or other materials known to pull away from a skin sealant. The skin sealant 12 in the reservoir 24 is positioned against the polymer film 42. When applied to the incision site, the skin sealant 12 extends at least to an outer edge of the defined reservoir 24, and desirably beyond (not shown).

The combined drape 10 may be provided with several variations: one with the polymer film 42 sealed to the incise drape 14 in the reservoir 24, and one without. The polymer film 42 coupled to the incise drape 14 in the reservoir 24 permits the skin sealant 12 to remain on and about the incision site even after the incise drape 14 of the combined drape 10 is removed. In addition, any portions of the skin sealant 12 which extend beyond the area of the polymer film 42 will be substantially removed with the incise drape 14 as well. When the combined drape 10 is provided without the polymer film 42 positioned against the reservoir of the incise drape 14, it is understood that the combined drape 10 (incise drape 14 and a substantial portion of the skin sealant 12) is removed together. Further, as previously described, the combined drape 10 may be provided with a porous film 36 included with the skin sealant 12 to provide a more evenly placed skin sealant 12 within the reservoir 24 and when applied to the prepped skin 38. Alternatively, the combined drape 10 may be provided without the porous film 36 included with the skin sealant 12. In a further alternative, the porous film 36 may be provided as a separate element or component in a package, and its use or non-use may be decided at the time the package is opened and the combined drape 10 is utilized.

The combined drape 10 may be provided in a kit or package 46, as illustrated in FIG. 17, which provides instructions 48 for use. Such instructions 48 may be provided separately, as shown, or may be printed on the package 46 (not shown). Such instructions 48 provide guidance as to the components of the combined drape 10, the selection and/or sequence of their use and/or removal, and indications for use or non-use, so that the combined drape 10 and the components thereof are utilized in an appropriate and desired manner.

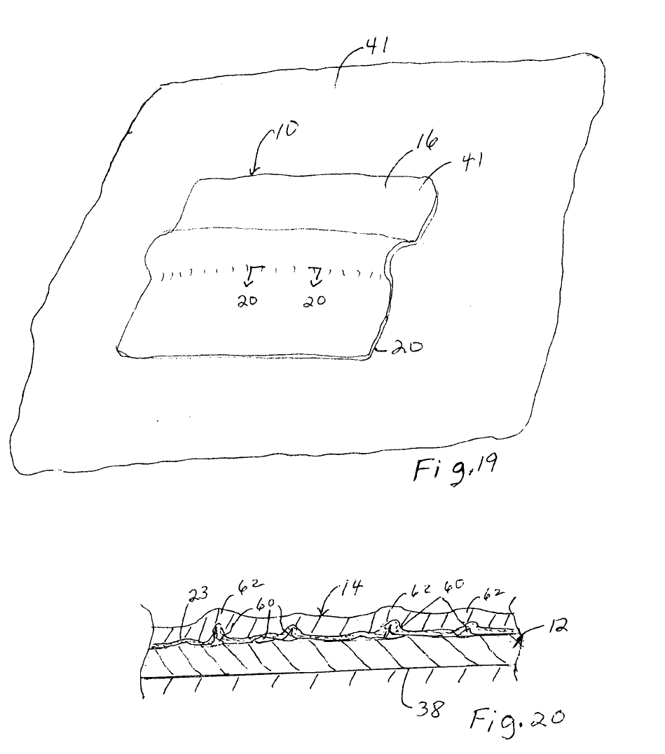

In another embodiment of the present invention, the combined drape 110 is provided in a package 146, as illustrated in FIG. 18. The combined drape 110 is provided as an incise drape 114 and the skin sealant 12 is provided separately, in a container 150. The container 150 may comprise an applicator, a tub, a soft package, or any appropriate storage and/or application vessel.

In this embodiment, the incise drape 114 may have one or a combination of any of the previously described features provided therewith. Alternatively, however, the incise drape 114 may be covered across at least substantially its entirety by a peel strip 122, or the incise drape may be provided without a peel strip and rolled, folded, and so forth, to protect its adhesive lower surface 118 (not shown).

The incise drape 14 may include one or any combination of the features already discussed (i.e., peel strips and/or portions of the incise drape formed in such a manner as to provide at least a portion of a reservoir 24). Alternatively, the incise drape 114 may be provided without a reservoir. The skin sealant 12 is applied to the incise drape 114 after at least a portion of the peel strip 122 is removed, or after a portion of the adhesive lower surface 18 of the incise drape 114 is exposed. The skin sealant 12 and/or the incise drape 114 may or may not form a reservoir 24 upon the application of the skin sealant 12 to the incise drape 114.

In this embodiment, the package 146 may also contain a piece of polymer film 42 to place in the reservoir 24 or on the central area 26 of the incise drape 114 which is to receive the skin sealant 12, if it is desired to remove the incise drape 114 and leave the skin sealant 12 in place after the surgery is completed. In addition, a piece of clear porous film 36 may be provided separately to use along with the skin sealant 12 so that the skin sealant 12 is dispersed evenly across the reservoir 24 or central area 26 of the incise drape 114. Alternatively, the porous film 36 may be provided with the skin sealant 12 in the same container 150, and both may be applied to the incise drape 114 together.

The kit or package 146 may also provide instructions 148 for use. Such instructions 146 may be provided separately, as shown, or may be printed on the package 146 (not shown). Such instructions 148 provide guidance as to the separate components of the combined drape 110, the selection and/or sequence of their use and/or removal, and indications for use or non-use, so that the combined drape 110 and the components thereof are utilized in an appropriate and desired manner.

As illustrated in FIGS. 19 and 20, the combined drape 10, 110 enhances the advantages and reduces the disadvantages of each separate element, i.e., the incise drape 14, 114 and the skin sealant 12, used alone. When the incise drape 14, 114 is applied to the prepped skin 38, it often forms pockets of air 60, which may include microbial agents therein. This occurrence is very undesirable, and is contrary to the desired result of eliminating microorganisms from the incision site and surrounding area. The skin sealant 12 positioned between the prepped skin 38 and the incision drape 14, 114 fills and substantially eliminates the occurrence of air pockets 60. This is particularly important in view of contours that usually are present on or adjacent an incision, in which it is common to observe air pockets 60 formed between the skin 38 and the incise drape 14, 114 which must necessarily wrinkle 62 and/or stretch to follow such contours. Even when air pockets 60 remain trapped beneath the incise drape 14, 114, it is important to note that they are not adjacent the skin 38, where they can migrate to the incision, but are positioned adjacent the skin sealant 12, which has an anti-microbial within, and sealed thereto. Therefore, the combined drape 10 provides a cleaner, safer incision site.

The use of the incise drape 14, 114 with the skin sealant 12 removes the disadvantage of the skin sealant 12, which is unable to act to stabilize, support, and anchor the surrounding drapes relative to the incision site. Tape, and/or adhesive on the surrounding drapes, are usually used to adhere to the skin sealant 12. However, it is easier for tape or adhesive on the drapes to have breaches or openings in the connection therebetween which are not readily apparent, and which may provide contaminants to the incision site. Further, it is easier to destabilize the overall surgical site when separate drapes can fail to remain adhered to the skin sealant 12. Again, this occurrence can result in contamination to the incision site. The use of the incise drape 14, 114 over the skin sealant 12, and its use to uniformly adhere and connect to the surrounding drapes, provides greater stability to the entire set of surgical drapes, thereby reducing the opportunity for contamination to the incision site.

The use of an incise drape with a skin sealant appears time consuming, more expensive, and redundant. That is, both are provided to do essentially the same job: cover an incision and the surrounding area. Therefore, this redundancy has been seen as a legitimate disadvantage, and each was used separately. And, the disadvantages of each remained a problem to be solved. Only after experimental, confidential use was it discovered that the incise drape and the skin sealant together created a synergy and cooperation. It was recognized that each substantially overcame the disadvantages of the other.

While the present invention has been described in connection with certain preferred embodiments it is to be understood that the subject matter encompassed by way of the present invention is not to be limited to those specific embodiments. On the contrary, it is intended for the subject matter of the invention to include all alternatives, modifications and equivalents as can be included within the spirit and scope of the following claims.

What is claimed is:

1. A combination drape, comprising:
a skin sealant; and
an incise drape,
wherein the skin sealant is disposed on the incise drape and the combined skin sealant and incise drape is applied to a patient's skin such that the skin sealant is positioned adjacent the skin and the incise drape is positioned over the skin sealant and the skin, wherein the skin sealant substantially fills contours of the incise drape positioned thereover such that any air pockets and wrinkles positioned between the incise drape and the skin sealant are sealed and isolated from the skin.

2. The combination drape of claim 1, wherein the skin sealant is maintained in a sealable condition on the cover by a cover extending thereover.

3. The combination drape of claim 1, wherein the incise drape includes at least one peel strip.

4. The combination drape of claim 3, wherein the at least one peel strip cooperates to form a reservoir in a central area of the incise drape, and the reservoir is configured to hold the skin sealant.

5. The combination drape of claim 1, wherein the skin sealant is contained within a container before it is disposed in the reservoir.

6. The combination drape of claim 1, wherein the incise drape includes a central area, and the skin sealant is disposed in the central area.

7. The combination drape of claim 6, wherein a reservoir is formed in the central area, and wherein a polymer film is positioned in the reservoir and against the drape before the skin sealant is disposed in the central area.

8. The combination drape of claim 1 further comprising a polymer film.

9. The combination drape of claim 8, wherein the polymer film is positioned in a central area of the incise drape.

10. The combination drape of claim 8, wherein the polymer film is positioned in a reservoir provided on the incise drape.

11. The combination drape of claim 1 further comprising a porous film.

12. The combination drape of claim 11, wherein the skin sealant includes the porous film.

13. The combination drape of claim 11, wherein the porous film is positioned with the skin sealant in a reservoir formed in the incise drape.

14. The combination drape of claim 11 wherein the porous film is provided in a container holding the skin sealant.

15. The combination drape of claim 1, further comprising a container containing the skin sealant.

16. The combination drape of claim 1, wherein the combination drape is contained in a package.

17. The combination drape of claim 16 wherein the package includes instructions.

18. A kit for draping an incision site, comprising:
a combination drape including
skin sealant;
an incise drape; and
instructions,
wherein the skin sealant is disposed on the incise drape and the combined skin sealant and incise drape is applied to a patient's skin such that the skin sealant is positioned adjacent the skin and the incise drape is positioned over the skin sealant and the skin, wherein the skin sealant substantially fills contours of the incise drape positioned thereover such that any air pockets and wrinkles positioned between the incise drape and the skin sealant are sealed and isolated from the skin, and
wherein the instructions provide guidance on the selection and sequence of use of items in the kit.

19. The kit of claim 18, wherein the skin sealant is maintained in a sealable condition on the cover by a cover extending thereover.

20. The kit of claim 18, wherein the incise drape includes at least one peel strip.

21. The kit of claim 20, wherein the at least one peel strip cooperates to form a reservoir in a central area of the incise drape, and the reservoir is configured to hold the skin sealant.

22. The kit of claim 18, wherein the skin sealant is contained within a container before it is disposed in the reservoir.

23. The kit of claim 18, wherein the incise drape includes a central area, and the skin sealant is disposed in the central area.

24. The kit of claim 23, wherein a reservoir is formed in the central area, and wherein a polymer film is positioned in the reservoir and against the drape before the skin sealant is disposed in the central area.

25. The kit of claim 18 further comprising a polymer film.

26. The kit of claim 25, wherein the polymer film is positioned in a central area of the incise drape.

27. The kit of claim 25, wherein the polymer film is positioned in a reservoir provided on the incise drape.

28. The kit of claim 18 further comprising a porous film.

29. The kit of claim 28, wherein the skin sealant includes the porous film.

30. The kit of claim 28, wherein the porous film is positioned with the skin sealant in a reservoir formed in the incise drape.

31. The kit of claim 28 wherein the porous film is provided in a container holding the skin sealant.

32. The kit of claim 18, further comprising a container containing the skin sealant.
